(19) United States
(12) Patent Application Publication
(10) Pub. No.: US 2024/0101515 A1
(43) Pub. Date: Mar. 28, 2024

(54) SYNTHESIS OF 3-NITRO-N-(2,2,2-TRIFLUOROETHYL)-4-PYRIDINAMINE

(71) Applicant: JANSSEN SCIENCES IRELAND UNLIMITED COMPANY, Co Cork (IE)

(72) Inventors: Hongyu TAN, Shanghai (CN); Licheng HAN, Shanghai (CN)

(21) Appl. No.: 17/754,588

(22) PCT Filed: Oct. 29, 2020
(86) PCT No.: PCT/EP2020/080379

§ 371 Date: Apr. 6, 2022

(30) Foreign Application Priority Data
Oct. 30, 2019 (WO) PCT/CN2019/114253

(51) Int. Cl. C07D 213/74

(52) U.S. Cl. CPC C07D 213/74

(57) ABSTRACT

The present invention relates to a chemical synthesis route for preparing 3-nitro-N-(2,2,2-trifluoroethyl)-4-pyridinamine which compound can be used as an intermediate compound in the synthesis of RSV inhibiting imidazopyridines or imidazopyrimidines.

The present invention relates to a chemical synthesis route for preparing 3-nitro-N-(2,2,2-trifluoroethyl)-4-pyridinamine which compound can be used as an intermediate compound in the synthesis of RSV inhibiting imidazopyridines or imidazopyrimidines.

### BACKGROUND

Respiratory syncytial virus is a major cause of acute lower respiratory tract infection in young children, immunocompromised adults, and the elderly. Intervention with small-molecule antivirals specific for respiratory syncytial virus presents an important therapeutic opportunity, but no such compounds are approved today.

3-Nitro-N-(2,2,2-trifluoroethyl)-4-pyridinamine is a compound that can be used as a building block in the synthesis of RSV inhibiting imidazopyridines or imidazopyrimidines such as those disclosed in WO-01/95910.

### PRIOR ART

WO-01/95910 discloses on page 65 the hydrochloric acid salt of 3-nitro-N-(2,2,2-trifluoroethyl)-4-pyridinamine which is prepared from 4-methoxy-3-nitro-pyridine as depicted below:

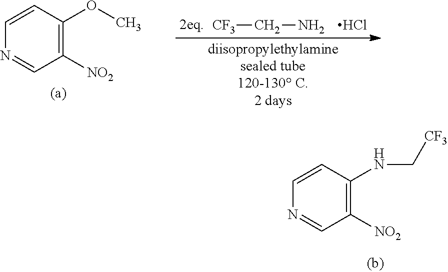

### DESCRIPTION OF THE INVENTION

It has now been found that 3-nitro-N-(2,2,2-trifluoroethyl)-4-pyridinamine can be prepared conveniently by treating 4-methoxy-3-nitro-pyridine with 2,2,2-trifluoroethylamine in the presence of an aqueous solution comprising an acid under heating.

For large scale production this new synthesis method avoids the use of a high-pressure reaction vessel (i.e. the upscaled version of a sealed tube) and reduced reaction time from 2 days to less than one day (typically between 10 to 16 hours).

The present invention provides a process for preparing 3-nitro-N-(2,2,2-trifluoroethyl)-4-pyridinamine of formula (b) by treating 4-methoxy-3-nitro-pyridine of formula (a) with 2,2,2-trifluoroethylamine in the presence of an aqueous solution comprising an acid under heating.

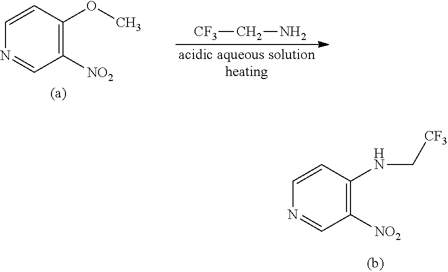

The acid used can be any inorganic or organic acid that is soluble in water and is capable of protonating the pyridine moiety to activate the pyridine ring for nucleophilic substitution. Inorganic acids are selected from hydrochloric acid, hydrobromic acid, sulfuric acid, phosphoric acid, boric acid, nitric acid, and the like. Organic acids are preferably carboxylic acids selected from formic acid, acetic acid, p-toluenesulfonic acid, citric acid, oxalic acid, malonic acid, succinic acid, lactic acid, and the like. A suitable organic acid is citric acid, or citric acid monohydrate, which has a low cost, low toxicity and easy to handle. The amount of organic acid can be any amount between 1 mole to 5 mole with respect to the amount of 4-methoxy-3-nitro-pyridine. In practice the amount of organic acid ranges from 1.2 to 3 mole and a preferred range is 1.2 to 1.5.

The amount of 2,2,2-trifluoroethylamine can be any amount between 1 mole to 5 mole with respect to the amount of 4-methoxy-3-nitro-pyridine. In practice the amount of 2,2,2-trifluoroethylamine ranges from 1.1 to 3 mole.

The reaction may conveniently be carried out at a temperature ranging between room temperature and the reflux temperature of the reaction mixture. In practice the temperature can range from room temperature to 90° C., or from 30° C. to 80° C., or from 40° C. to 70° C.

Upon completion of the reaction, the desired 3-nitro-N-(2,2,2-trifluoroethyl)-4-pyridinamine can be isolated from the reaction mixture by extraction with an organic solvent.

### Example: Synthesis of 3-nitro-N-(2,2,2-trifluoroethyl)-4 pyridinamine

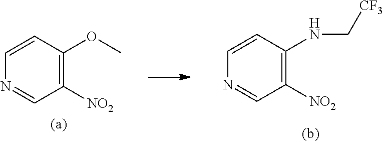

Citric acid monohoydrate (295.0 g) is dissolved in water (370.0 g) and 4-methoxy-3-nitropyridine (179.0 g) is then added followed by 2,2,2-trifluoroethylamine (348.0 g). The mixture is stirred at 50° C. until complete conversion (10-16 hours). After cooling to room temperature, 2-MeTHF (1250 mL) is added and the mixture is stirred for 1 hour and the phases are then separated. The aqueous phase is re-extracted with 2-MeTHF (530 mL). The combined organic layers are washed with 7% aqueous NaHCO₃ solution (890 g) and finally with water (903 g). The organic layer is evaporated to dryness yielding 3-nitro-N-(2,2,2-trifluoroethyl)-4-pyridinamine (233.7 g, 91% yield).

¹H NMR (400 MHz, DMSO-d₆) δ ppm 4.28-4.47 (m, 2H); 7.28 (d, J=6.27 Hz, 1H); 8.38 (d, J=6.02 Hz, 1H); 8.63 (br t, J=6.65 Hz, 1H); 9.08 (s, 1H)

¹³C NMR (101 MHz, DMSO-d₆) δ ppm 43.15 (q, J=33.0 Hz); 109.30 124.05; 126.84; 130.70; 148.55 (d, J=4.4 Hz); 153.68

1. A process for preparing 3-nitro-N-(2,2,2-trifluoroethyl)-4-pyridinamine of formula (b) 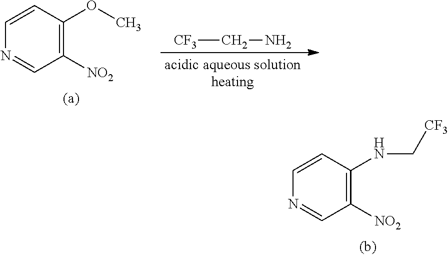
comprising treating 4-methoxy-3-nitro-pyridine of formula (a) with 2,2,2-trifluoroethylamine in an aqueous solution comprising an acid under heating.

2. The process of claim 1, wherein the acid is an inorganic acid or an organic acid.

3. The process of claim 2, wherein the inorganic acid is selected from the group consisting of hydrochloric acid, hydrobromic acid, sulfuric acid, phosphoric acid, boric acid, and nitric acid.

4. The process of claim 2, wherein the organic acid selected from the group consisting of formic acid, acetic acid, p-toluenesulfonic acid, citric acid, oxalic acid, malonic acid, succinic acid, and lactic acid.

5. The process of claim 1, wherein the organic acid is citric acid.

6. The process of claim 1, wherein the reaction mixture is heated to a temperature ranging from room temperature to the reflux temperature of the reaction mixture.

7. The process of claim 1, wherein the temperature ranges from 30° C. to 80° C.

8. The process of claim 1, wherein the temperature ranges from 40° C. to 70° C.

9. The process of claim 2, wherein the amount of organic acid ranges from 1 mole to 5 mole and the amount of 2,2,2-trifluoroethylamine ranges from 1 to 3 mole.
